(19) United States
(12) Patent Application Publication
(10) Pub. No.: US 2022/0184228 A1
(43) Pub. Date: Jun. 16, 2022

(54) METHODS OF PROMOTING LONG-TERM SURVIVAL OF PATIENTS WITH ADVANCED CHEMOTHERAPY-RESISTANT MALIGNANCIES

(71) Applicants: Erlinda M. Gordon, Santa Monica, CA (US); Frederick L. Hall, Carmel Valley, CA (US)

(72) Inventors: Erlinda M. Gordon, Santa Monica, CA (US); Frederick L. Hall, Carmel Valley, CA (US)

(21) Appl. No.: 17/603,268

(22) PCT Filed: Apr. 13, 2020
(86) PCT No.: PCT/US2020/027995

§ 371 Date: Oct. 12, 2021

Related U.S. Application Data
(60) Provisional application No. 62/833,363, filed on Apr. 12, 2019.

(51) Int. Cl. A61K 48/00; A61K 47/64; A61P 35/00; C07K 14/47; C12N 15/86

(52) U.S. Cl. CPC A61K 48/005; A61K 47/64; C12N 15/86; C07K 14/4738; A61P 35/00

(57) ABSTRACT

Disclosed are methods for treating an individual with a chemotherapy-resistant primary and/or secondary malignancy that comprise administering a therapeutically effective amount of a tumor-targeted retrovector encoding a cytocidal dominant-negative Cyclin G1 construct, such as DeltaRex-G, to the individual. Also disclosed are methods in which DeltaRex-G, either alone or in conjunction with other cancer therapies and/or treatments, may be administered to ameliorate or eliminate the life-threatening effects of metastatic cancer.

### CROSS-REFERENCE TO RELATED APPLICATIONS

This application claims the benefit of U.S. Provisional Application No. 62/833,363, filed on Apr. 12, 2019, which is hereby incorporated by reference in its entirety.

### TECHNICAL FIELD

This disclosure relates to methods of treating patients with cancer. More specifically, this disclosure relates to methods of promoting long-term survival in patients with advanced chemotherapy-resistant cancer. This disclosure also relates to methods of identifying and eradicating cancer stem cells using novel cancer biomarkers that are major effectors of cancer stem cell activation and signaling. This disclosure also relates to methods of restoring drug sensitivity, breaking immune anergy, and restoring tumor surveillance function without evoking systemic and organ toxicity.

### BACKGROUND

Cancer, which is the second leading cause of death world-wide, is an enormous global health burden, touching every region and socioeconomic group. In 2018, the American Cancer Society estimates that there were 17.0 million new cases of cancer diagnosed around the world and 9.5 million cancer deaths. American Cancer Society (2018), Global Cancer Facts & Figures 4th Edition. In the U.S. alone, there will be an estimated 1,762,450 new cancer cases diagnosed and 606,880 cancer deaths in 2019. American Cancer Society, Cancer Facts & Figures 2019. Moreover, the global cancer burden is growing at an alarming pace. It is predicted that in 2040, there will be about 27.5 million new cancer cases and 16.3 million cancer deaths.

Cancer treatment typically includes one or a combination of surgery, radiation therapy, chemotherapy, hormone therapy, targeted therapy, and/or immunotherapy. Determining a cancer patient's treatment options will usually depend on several factors such as the tumor type and stage, whether the patient has already had a particular type of therapy (to which the cancer cells are now resistant), and the patient's health.

“Chemotherapy” refers to treatments that use one or more drugs to kill cancer cells. Chemotherapeutic agents, however, do not only kill cancer cells. Such agents may also kill other cells such as those that line a patient's mouth and intestines and certain immune system cells.

The inherent toxicities of many approved chemotherapeutic agents are a result of the non-specific nature of drug distribution, which also damages normal tissues and organs. Chemotherapies and biologics are distributed widely to both target and non-target organs. Such anticancer agents generally require high plasma levels, which kill both cancer cells and healthy cells and thus can be dangerous. This non-targeted biodistribution often results in untoward systemic toxicities which negatively impact the treatment regimen and outcome.

Cancer cells from a tumor treated with chemotherapy frequently become resistant to the particular chemotherapeutic agent. Without being bound by any particular theory, some cancer cells within a population may carry mutations that render such cells less susceptible to the chemotherapeutic agent. Alternatively, cancer cells may undergo epigenetic changes that alter gene expression such that the cells are less susceptible to the chemotherapeutic agent. Cancer cells that are resistant to chemotherapy may cause a relapse, which is a reoccurrence of the cancer. Such recurring cancer may spread to other organs. Metastatic cancer has been associated, hitherto, with an invariably fatal outcome. Another aspect of the systemic toxicities arising from some chemotherapy is the occurrence of secondary malignancies, which can be of a different type and affect different tissues than the initial malignancy. For example, chemotherapies that target cancer cell DNA, such as alkylating agents and/or topoisomerase II inhibitors, are often associated with the occurrence of secondary malignancies, often with a latency of months to years after the initial treatment. These malignancies tend to be highly resistant to therapy and have quite low survival rates.

### DESCRIPTION OF THE FIGURES

The written disclosure herein describes illustrative embodiments that are non-limiting and non-exhaustive. Reference is made to certain of such illustrative embodiments that are depicted in or related to the figures described herein.

FIG. 1 shows a structure/function analysis of the Cyclin-G1 gene (left) and the dominant negative G1 in accordance with an embodiment (right).

FIG. 2 shows a diagram illustrating the interaction of various molecular components of CCNG1 oncogenic pathways.

FIG. 3 shows immunohistochemical analyses of a leiomyosarcoma metastatic to a pelvic lymph node, where (A) shows an H&E stained tissue section from the resected nodule; and (B) and (C) show histological analyses of levels of CCNG1 and Ki-67, respectively. Scale bar=200 μm.

FIG. 4 provides graphs that illustrate significant regression of bone metastases (shown in FIG. 4A by FDG avidity measured by PET-CT scan), associated with fluctuating CA-15 levels (shown in FIG. 4B) with the lowest levels achieved during treatment with the GeneVieve Protocol, which is described in Ignacio et al. (2018), Clinics in Oncology 3:1537.

FIG. 5 provides bone scans that show progressive regression of bone metastases during treatment with the GeneVieve Protocol (A) before treatment; (B): after DeltaRex-G (Former Name: Rexin-G) treatment; (C): after completion of treatment protocol. The left and right images in each panel show anterior and posterior views, respectively).

FIG. 6 shows histological analyses of a 6 mm metastatic tumor nodule remaining after a three-week course of DeltaRex-G in which two other metastatic tumor nodules were eradicated. (A) and (D) show hematoxylin and eosin (H&E) stained tissue sections from the resected residual nodule; (B) and (C) show histological analyses of levels of fibrosis and CA 15.3, respectively; (E) and (F) show histological analyses for infiltration of CD45+ macrophages and CD8+ killer T cells, respectively. DeltaRex-G enhances immune cell trafficking in the tumor microenvironment as evidenced by presence of CD45+ M1 and M2 macrophages and CD8+ killer T cells. These data indicate that DeltaRex-G is an immune modulator and may be used in conjunction with immune check point inhibitors such is CTLA4, PD1/PDI1 inhibitors.

FIG. 7 illustrates tumor regression and CA-19.9 levels during treatment with DeltaRex-G in a patient (Patient 018). (A) Percentage changes in tumor size (longest diameter [LD]) of metastatic hepatic and lymph node sub-peritoneal lesions are individually plotted on the vertical axis, as a function of time from the beginning of DeltaRex-G treatment, plotted on the horizontal axis. (B) Serum levels of tumor marker CA-19.9 (U/mL) are plotted on the vertical axis, as a function of time from the beginning of DeltaRex-G treatment, plotted on the horizontal axis.

FIG. 8 provides histopathological findings in the lung metastases of a patient with chemotherapy-resistant osteosarcoma after DeltaRex-G treatment. Representative hematoxylin and eosin (H&E) stained tissue sections from resected residual lung nodules are provided.

FIG. 9 illustrates various tumor parameters of a patient monitored over time. (A) Individual tumor volume measurements over time. Tumor volumes of individual target lesions (measured by CT imaging), in mm³, were calculated using O′Reilly's formula (length×width²×0.52). (B) Individual tumor density measurements over time. Tumor densities of individual target lesions, in HU, were calculated by radiographic image analysis. (C) Individual FDG avidity measurements over time. Avidities of individual target lesions for 18-FDG (SUVₘₐₓ) were measured in individual target lesions using a PET/CT scan. CT, computed tomography; PET, positron emission tomography; HU, Hounsfeld units; FDG, fluorodeoxyglucose; SUVₘₐₓ, maximum standardized uptake value.

### DETAILED DESCRIPTION

The present disclosure is generally related to methods of treating individuals with cancer, especially chemotherapy-resistant and/or metastatic cancer. In some embodiments, the disclosure provides methods of treating an individual with a chemotherapy-resistant malignancy by administering a therapeutically effective amount of an inhibitor of the activity or downstream effects of the CCNG1 gene, or more particularly a tumor-targeted retrovector that encodes a cytocidal dominant-negative cyclin G1 construct, to the individual. This tumor-targeted retrovector may be DeltaRex-G. DeltaRex-G (former names include Rexin-G and Mx-dnG1) is a tumor-targeted retrovector that encodes a cytocidal dominant negative cyclin G1 (“dnG1”) construct. See, e.g., Al-Shihabi et al. (2018), Molecular Therapy: Oncolytics 11:122-126; Kim et al. (2017), Molecular and Clinical Oncology 6:861-865; Chawla et al. (2016), Sarcoma Research International 3(1):1024; Chawla et al. (2019), Molecular Therapy: Oncolytics 12:156-167; Ignacio et al. (2018), Clinics in Oncology 3:1537; Chawla et al. (2009), Molecular Therapy 17(9):1651-7. Without being bound by any particular theory, DeltaRex-G nanoparticles actively seek-out the cryptic “signatures of disease” (“SIG”) proteins within the tumor microenvironment (“TME”) and thereby accumulate in tumors, delivering the “killer gene” selectively to tumor cells.

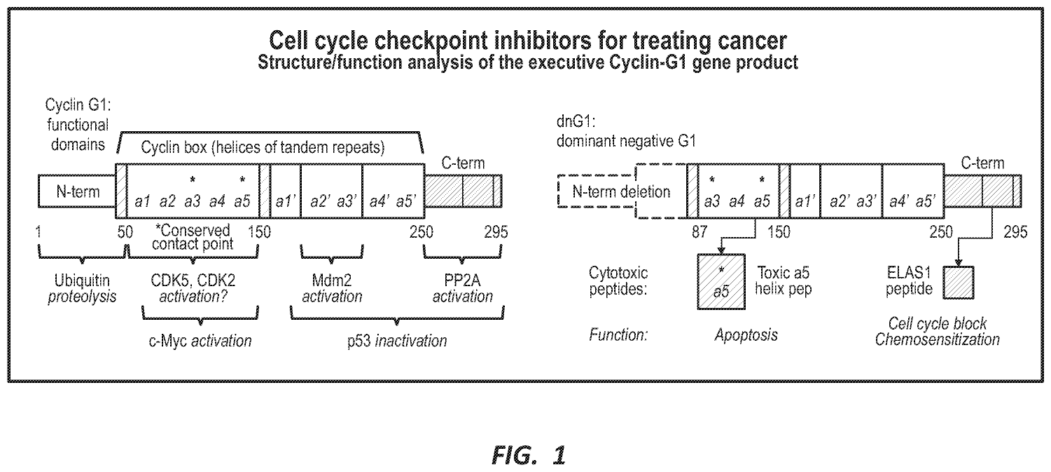

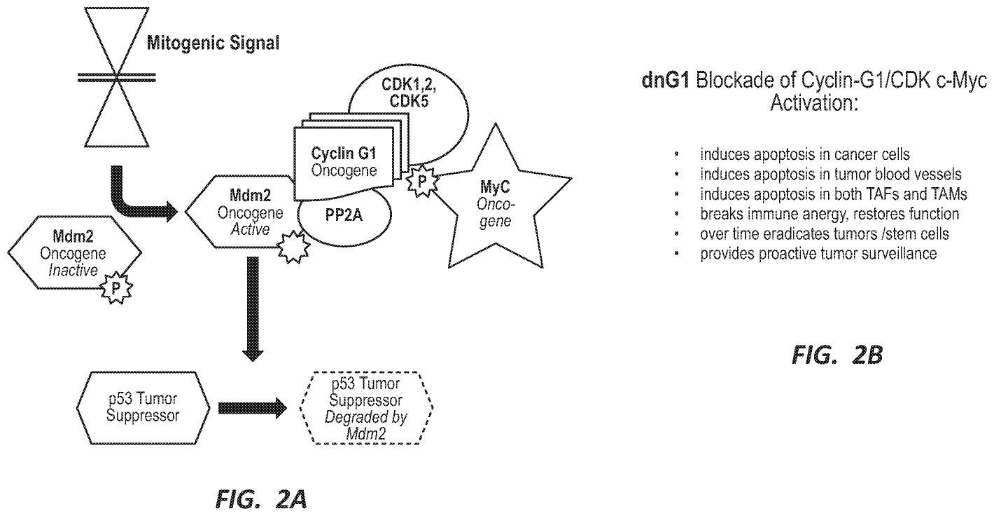

A structural/functional diagram of the cytocidal dnG1 construct is shown in FIG. 1. The construct is devoid of the ubiquitinated N-terminus (proteolytic processing), as well as the first two helical segments (α1 and α2) of the definitive cyclin box, characteristically arrayed in cyclins as a tandem set of helical segments, including two highly-conserved residues essential for cyclin-dependent kinase (CDK) binding1. The cytocidal dnG1 protein, which induces apoptosis in proliferative cells, retains the presumptive CDK contact points (Helix α3*, α5*) and the complete structural domains attributed to PP2A, β′ and Mdm2 binding. Recently, new therapeutic synthetic peptides (e.g., ELAS1 and α5 Helix peptides, see FIG. 1) derived from structures and/or homologous interfaces contained within the dnG1 protein are themselves reported to induce cell cycle blockade and apoptosis, respectively.

The words “treating” and “treatment” or “therapy” as used herein have their usual meanings in medical science, that is, “treating” means the management and care of a patient to cure or alleviate a disease or disorder or the symptoms thereof. A treatment or therapy may achieve a “cure,” that is, a complete and permanent remission of a cancer, but it need not be a cure. Treatment may be undertaken to alleviate symptoms, for example, to decrease tumor size, the number and location of metastases, or the physiological effects of tumor burden. Treatment may lead to temporary remission or render the tumor more amenable to other therapeutic options (such as surgery, radiation, or treatment with a different therapeutic agent or combination of agents). It should also be noted that use of these terms is not meant to exclude other steps that may be necessary or desirable for the management and care of a cancer patient but that are not recited in the methods described in this disclosure, e.g., use of IV fluids for the patient's hydration or use of medications to treat pain.

Mitogenic signal transduction cascades from extracellular growth-factor receptor-mediated events through proline-directed protein phosphorylation as a major regulatory theme, which can be sub-divided enzymatically into mitogen-activated protein kinases/extracellular signal-regulated kinases (MAPKs/ERKs) and Cyclin-dependent protein kinase (CDK) complexes which govern the progressive phases of the cell division cycle. As the name implies, CDKs are cell cycle control enzymes whose activation requires the induction and expression of genes for cyclins—relatively unstable positive-acting regulatory subunits of CDKs, often exhibiting cyclically oscillating levels. Some of these genes are oncogenes, encoding a transforming cyclin oncoprotein that physically escorts its otherwise undiscerning and inactive CDK partner(s) to an executive biochemical locus regulating gene expression, and hence exerting executive cell cycle control.

The protein Cyclin G1, the gene product of CCNG1, is a prime factor in initiating a state of cell competence in which cell viability is favored over DNA fidelity and whereby cancer stem cells remain competent to hide from immunological surveillance, thrive, and proliferate. The core elements of the cellular pathways implicated in this phenomenon are shown in Panel A of FIG. 1. Cyclin G1 physically binds to a major cellular serine/threonine protein phosphatase subunit designated 2A (PP2A), thereby targeting this otherwise undiscerning phosphatase to dephosphorylate—and thereby activate—a Cyclin G1-targeted protein, MDM2 (an oncogene product). The MDM2 protein in turn targets, inhibits, and degrades the p53 tumor suppressor, an important substrate in the normal regulation of the cell division cycle by promoting DNA fidelity through cell cycle arrest, DNA repair, apoptosis, and cellular senescence. In the absence of p53 tumor suppression, CCNG1/Cyclin G1 drives a sustained state of cell competence as observed in cancer stem cells. In accordance with some embodiments, therapeutic methods comprising the administration of CCNG1 inhibitors, or more particularly DeltaRex-G, can inhibit or arrest tumorigenic outcomes of this competence. Without being bound to a particular theory, it is believed that CCNG1 inhibitors may accomplish this through a number of mechanisms, such as are shown for DeltaRex-G in Panel B of FIG. 1 by way of example.

As discussed in the Examples below, the methods disclosed herein may be used to treat individuals with any of several types of chemotherapy-resistant and/or metastatic malignancies. In some embodiments, the chemotherapy-resistant malignancy has acquired a resistance to a chemotherapeutic agent that has been employed to treat that malignancy. In some embodiments, the chemotherapy-resistant malignancy is a second malignancy, the occurrence of which is induced by chemotherapy employed against an earlier-occurring malignancy. The methods disclosed herein may be used to prevent or reduce the occurrence of chemotherapy-induced second malignancies.

In some embodiments, the malignancy may be a soft tissue sarcoma, osteosarcoma, myeloma, or pancreatic adenocarcinoma. In some embodiments, the malignancy may be a metastatic pancreatic adenocarcinoma, osteosarcoma, malignant peripheral nerve sheath tumor, intraductal carcinoma of breast, B-cell lymphoma, chondroblastic osteosarcoma of maxilla, glioblastoma, or other metastatic malignancy.

In some embodiments, methods disclosed herein may further comprise the step of administering one or more additional cancer therapies, for example, immunotherapy. In certain embodiments, methods may further comprise administering a tumor-targeted retrovector encoding a granulocyte macrophage colony-stimulating factor (GM-CSF) protein. The GM-CSF protein sequence may be equivalent to a human GM-CSF protein sequence, or may be altered at one or more amino acid positions. The GM-CSF protein sequence may be equivalent to a non-human animal GM-CSF protein sequence or derivative thereof. In some embodiments, the tumor-targeted retrovector encoding a GM-CSF protein is DeltaVax (former name: Reximmune-C), which encodes genes for GM-CSF and herpes simplex virus thymidine kinase (HSV-TK). Without being bound by any particular theory, HSV-TK is a phosphotransferase that phosphorylates certain nucleoside analogs (e.g., ganciclovir). These phosphorylated nucleoside analogs can inhibit DNA replication.

In certain embodiments, the CCNG1 inhibitor is administered intravenously. In other embodiments, the CCNG1 inhibitor may be administered using another route of administration, e.g., by subcutaneous, intramuscular, intradermal, transdermal, intrathecal, intracerebral, intraperitoneal, intranasal, epidural, pulmonary, intravitreal, or oral routes. Administration may be immediate or rapid, such as by injection, or carried out over a period of time, such as by infusion or administration of controlled or delayed release formulations. In some embodiments, administration is carried out by continuous infusion, particularly by using an infusion pump. In certain cases, such as when the treated individual is ambulatory, a portable infusion pump may be used.

In certain embodiments, a tumor-targeted retrovector encoding a cytocidal dominant-negative cyclin G1 construct is administered at a therapeutically effective dose. In some embodiments, the vector is administered at a dose of greater than about 10¹⁰ colony forming units (cfu). As discussed below with respect to various embodiments, the vector may be also be introduced at a dose of greater than about 10¹¹ cfu or other suitable dose based on the particular parameters of the disease presentation in the individual and/or one or more selected clinical endpoints.

The tumor-targeted retrovector encoding a cytocidal dominant-negative cyclin G1 construct may be administered more than once to an individual in need thereof. In some embodiments, the retrovector may be administered at least two times per week for a period of at least three weeks. In other embodiments, the retrovector may be administered at least three times per week for a period of at least four weeks. In other embodiments, an individual with metastatic cancer may be periodically administered the retrovector until there is regression of metastases. Regression of metastases may be determined using conventional methods for such analyses. For example, bone metastases may be evaluated by PET-CT scan or by periodically monitoring the level of one or more tumor biomarkers.

The present disclosure also provides methods of treating an individual with a chemotherapy-resistant malignancy comprising the steps of (1) administering a therapeutically effective dose of a first tumor-targeted retrovector that encodes a cytocidal dominant-negative cyclin G1 construct to the individual at least three times per week for a period of at least four weeks, followed by a rest period of at least about two weeks; and (2) administering at least one therapeutically effective dose of a second tumor-targeted retrovector that encodes a GM-CSF gene to the individual.

The present disclosure also provides methods of treating an individual with a metastatic tumor that comprise the steps of (1) determining a first level (a “baseline”) of tumor metastases in the individual; (2) administering one or more therapeutically effective doses of DeltaRex-G to the individual; (3) periodically monitoring tumor metastases in the individual; and (4) continuing the DeltaRex-G therapy by administering one or more additional therapeutically effective doses of DeltaRex-G to the individual until there is a significant regression of metastases.

In certain embodiments, the disclosure provides methods that comprise the steps of (1) identifying an individual with a chemotherapy-resistant malignancy and (2) administering a therapeutically effective amount of a tumor-targeted retrovector, wherein the retrovector encodes a cytocidal dominant negative cyclin G1 construct to the individual. In some embodiments, the tumor-targeted retrovector is DeltaRex-G. The malignancy may be any of several types of chemotherapy-resistant malignancies. In some embodiments, the malignancy may be a soft tissue sarcoma, osteosarcoma, pancreatic adenocarcinoma, or other chemotherapy-resistant malignancy. In certain embodiments, the chemotherapy-resistant malignancy is a therapy-induced secondary malignancy.

In some embodiments, the biomarkers and major effectors of the executive Cyclin G1 gene product may be analyzed by molecular profiling using DNA or RNA analysis of Cyclin G1 Functional Domains and used to generate inhibitors of such major effectors and oncogenic drivers.

In some embodiments, methods for improving the long-term survival of an individual with a metastatic tumor comprise the steps of (1) determining or having determined whether the metastatic tumor exhibits at least one indicator of cancer stem cell competence, or more particularly of CCNG1 overexpression, Myc amplification, or both; and (2) administering a high-dose, therapeutically effective amount of a CCNG1 inhibitor to the individual if the metastatic tumor exhibits at least one such indicator, or adding an alternative therapy if the tumor does not exhibit at least one such indicator. In certain embodiments, the CCNG1 inhibitor is a tumor-targeted retrovector encoding a cytocidal dominant-negative cyclin G1 construct. In particular embodiments, the retrovector is DeltaRex-G. In some embodiments, the CCNG1 inhibitor is administered for a treatment period of at least three weeks. In certain embodiments, the treatment period is up to 24 months. In certain embodiments, the duration of the treatment period is determined based the attainment of one or more selected clinical endpoints, where the CCNG1 inhibitor is administered until the individual's tumor burden has been substantially eliminated, until the individual goes into remission, until the metastatic tumor stops growing, or until there are no circulating cancer cells in the individual's blood.

It is contemplated that inhibition of the CCNG1 pathway can be effective to prevent secondary malignancies in an individual with cancer, such that the individual will not need further anti-cancer therapy after primary therapy has concluded. In some embodiments, methods for preventing occurrence of a secondary malignancy in an individual having a tumor load comprises the steps of (1) administering a CCNG1 inhibitor to the individual in a therapeutically effective total dose over a treatment period of up to 15 weeks and (2) administering no further anti-cancer therapy to the individual after the treatment period. In various embodiments the therapeutically effective dose is selected based on the mode of delivery and relevant parameters of the disease. For example, where the CCNG1 inhibitor is provided via a retrovector, the number of colony forming units in an effective dose may be based on the individual's tumor load, i.e., the number of cancer cells present in the individual. In some embodiments, the therapeutically effective total dose comprises about 700 cfu to about 1500 cfu multiplied by the tumor load, or more particularly from about 900 cfu to about 1200 cfu. In a particular embodiment, the therapeutically effective total dose comprises about 1000 cfu multiplied by the tumor load. In certain embodiments, the CCNG1 inhibitor is administered to the individual via continuous intravenous infusion for a period of time. In particular embodiments, continuous infusion is carried out for a period of from about 7 to about 15 days.

In some embodiments, methods of treating a therapy-induced secondary malignancy in an individual comprise the steps of (1) determining or having determined that the malignancy exhibits at least one indicator of cancer stem cell competence; and (2) administering a CCNG1 inhibitor to the individual in a therapeutically effective total dose over a treatment period of up to 15 weeks or until an endpoint is reached. In more particular embodiments, the treatment period is from 8 weeks to 12 weeks.

The indicator can be related to products of CCNG1 activity, such CCNG1 overexpression or Myc amplification. In some embodiments, the indicator is a chromosomal mutation, e.g. insertions, translocations and other rearrangements, such as are often seen in association with therapy-induced malignancies. For example, immunoglobulin genes such as light kappa (κ) chain genes (IGK), heavy chain genes (IGH), and light lambda (λ) chain genes (IGL) are often involved in chromosomal rearrangements in B-cell lymphomas. In certain embodiments, the indicator can be a chromosomal rearrangement comprising juxtaposition of an immunoglobulin gene and CCNG1. In particular embodiments, the chromosomal rearrangement comprises juxtaposition of IGH and CCNG1. In certain embodiments, the therapy-induced secondary malignancy is a myeloma.

In some embodiments, the treatment period is directed to achievement of a particular clinical endpoint. In certain embodiments the endpoint is a target level of reduction in or substantial elimination of the tumor load of the individual. In a particular embodiment, the endpoint is a reduction of at least 50% in a tumor load in the individual. In another particular embodiment, the endpoint is a reduction of at least 80% in a tumor load in the individual. In some embodiments, the endpoint is a reduction in an indicator of cancer stem cell competence, such as a biomarker associated with CCNG1 pathway activity. In certain embodiments, the endpoint is a target level of reduction in Myc activation in the malignancy. In a particular embodiment, the endpoint is a reduction of at least 50% in Myc activation.

Without being bound by a particular theory, one aspect of methods of treatment described herein is that constitutively competent cancer stem cells can be rendered nonproliferative, or quiescent, by strategic blockade of the CCNG1 pathway. Cancer stem cells can be characterized by assaying markers of their state. For example, standard immunohistochemistry approaches can be employed to characterize assay constitutive CCNG1 expression, where CCNG1 overexpression is an identifying character of cancer stem cells. Similar methodologies can be used to assay a proliferative index such as Ki-67 to determine the degree to which the constitutive competency of these cells has been disrupted by CCNG1 inhibition, rendering them quiescent. It is contemplated that a sufficient level of quiescence may be effective to prevent further occurrences of malignancy.

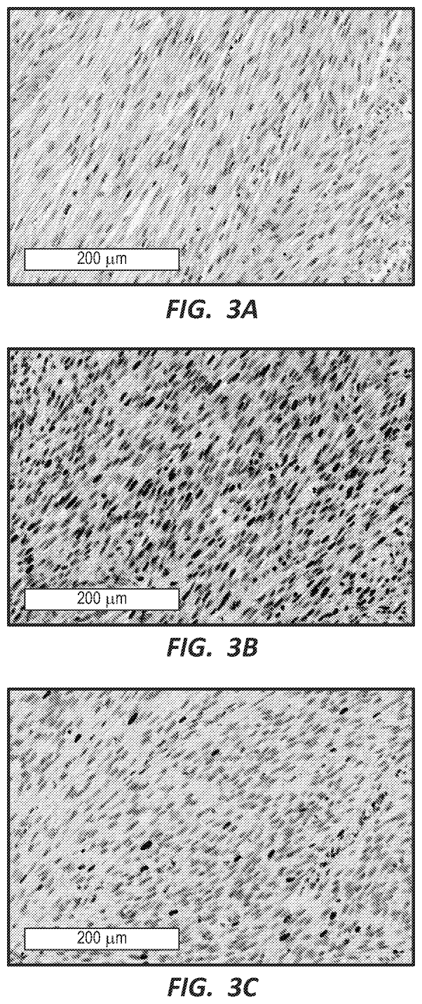

In certain embodiments, the disclosure provides methods of identifying cancer stem cells wherein the proto-oncogene CCNG1 is overexpressed in a metastatic tumor that is at the same time quiescent. In an aspect, overexpression and quiescence are evaluated relative to normal levels in a particular tissue or organ. For example, some tissues/organs (e.g., liver, heart, brain, kidneys, and muscle) normally exhibit little to no CCNG1 expression, while others exhibit higher levels (e.g., around 35% CCNG1+ cells in bone marrow, colon, and skin, and about 90% in tonsils and testicles). FIG. 3 shows an example of a leiomycosarcoma (smooth muscle tumor) where the presence of cancer stem cells is indicated by a high level (80%) of CCNG1+ cells, but the tumor is also quiescent. In some embodiments, methods for preventing occurrence of a secondary malignancy in an individual comprise the steps of (1) administering a CCNG1 inhibitor to the individual; (2) characterizing cancer stem cells in the individual to determine a quiescence level of the cancer stem cells; and (3) discontinuing anti-cancer therapy upon determining that the cancer stem cells have reached a quiescence endpoint. In some embodiments, the characterizing step comprises the steps of (1) measuring a CCNG1 expression level in a tissue sample from the individual to identify cells exhibiting overexpression of CCNG1 as cancer stem cells; (2) measuring a Ki-67 expression level in the tissue sample; and (3) comparing the Ki-67 expression level to the CCNG1 expression level to determine whether the quiescence endpoint has been reached. In an aspect, quiescence can be generally recognized where cancer stem cells are identified as being present in a tissue of interest, e.g. by CCNG1 overexpression, but the proliferative index is low. Accordingly, in some embodiments, the quiescence endpoint is a target ratio of CCNG1 expression to Ki-67 expression as measured in a tissue sample. In certain embodiments, the quiescence endpoint is a ratio of Ki-67 expression level to CCNG1 expression level of from about 2:1 to about 5:1. In certain embodiments, the quiescence endpoint is a target level of Ki-67 expression, such as less than about 10%.

In other embodiments, a method of arresting tumorigenesis in an individual comprises the steps of (1) identifying cancer stem cells exhibiting overexpression of CCNG1 or activation of a protein of the CCNG1 pathway and (2) administering a therapeutic agent that inhibits one or more molecular products of the CCNG1 pathway in an amount effective to render the cancer stem cells quiescent.

Without being bound by a particular theory, it is contemplated that the oncogenic and tumorigenic effects of dysregulated CCNG1 expression include, in addition to the suppression of p53-mediated apoptosis, the disruption of normal immune surveillance resulting in anergy, i.e. the ability of the immune system to be activated by tumor antigens is greatly compromised. In some embodiments, the disclosure provides methods of inducing apoptosis of cancer cells, breaking anergy and restoring immune tumor surveillance function. An aspect of the methods described herein is that these outcomes can be achieved without the toxic effects on bone marrow and other components of immune function that can be associated with other therapies. In particular embodiments, a method of breaking anergy to cancer cells by killing tumor associated malignant fibroblasts (TAFs), reducing extracellular matrix (ECM) in the tumor microenvironment (TME) and enabling entry of patient's own immune cells into the TME comprises administering a therapeutically effective amount of a tumor-targeted retrovector to the individual, wherein the retrovector encodes a cytocidal dominant-negative cyclin G1 construct. In other embodiments, a method of restoring or inducing proactive tumor surveillance function by killing cancer cells, tumor associated malignant fibroblasts (TAFs), and tumor associated macrophages (TAM) without bone marrow or immune suppression comprises administering a therapeutically effective amount of a tumor-targeted retrovector to the individual, wherein the retrovector encodes a cytocidal dominant-negative cyclin G1 construct.

### EXAMPLES

The following examples are for illustration only. In light of this disclosure, those of skill in the art will recognize that variations of these examples and other embodiments of the disclosed subject matter are enabled without undue experimentation.

### Example 1

### Induction of Sustained Ten-Year Remissions in Cancer Patients

DeltaRex-G tumor-targeted retrovector encodes a cytocidal dominant negative cyclin G1 construct and induced sustained 10-year remissions in patients with advanced chemotherapy-resistant soft tissue sarcoma, osteosarcoma, and pancreatic adenocarcinoma. Methods: This is an open label, single arm, dose-seeking study that incorporated a modification of the standard Cohort of 3 design combined with a Phase II efficacy phase. Safety analysis used the NIH CTCAE vs 3.0 for reporting adverse events, and efficacy analysis used RECIST v1.0, International PET criteria and Choi criteria.

Results: Twenty patients received escalating doses of DeltaRex-G i.v. from 8×10¹¹ cfu to 48×10¹¹ cfu/6-week cycle. Safety (n=20): Grade 1-2 treatment-related adverse events included chills (n=1), pruritus (n=2), dry skin (n=1), hot flush (n=1), dysgeusia (n=3); Grade 3 pruritic rash (n=1). No dose-limiting toxicity was observed, and no vector DNA integration, replication-competent retrovirus, nor vector-neutralizing antibodies were detected. Efficacy (n=17): By RECIST v1.0: There were 13 SD, 4PD; by PET/Choi Criteria: 3 PR, 11SD, 3PD. Combined median PFS by RECIST v1.0 was 3.0 months; combined median OS, 30 months with 1-year overall survival rate of 60%. Biopsy of residual tumor in one patient identified abundant CD35+ dendritic cells, CD8+ killer T cells, CD138+ plasma B cells, CD68+ macrophages and CD20+ B cells, suggesting a mature immune response. Two patients with pure bone metastases had >12-month PFS and OS and are the longest survivors. One patient is still alive 10 years later. See Tables 1-3.

Case 1: Long Term (10-Year) Survivor with Pure Bone Metastases:

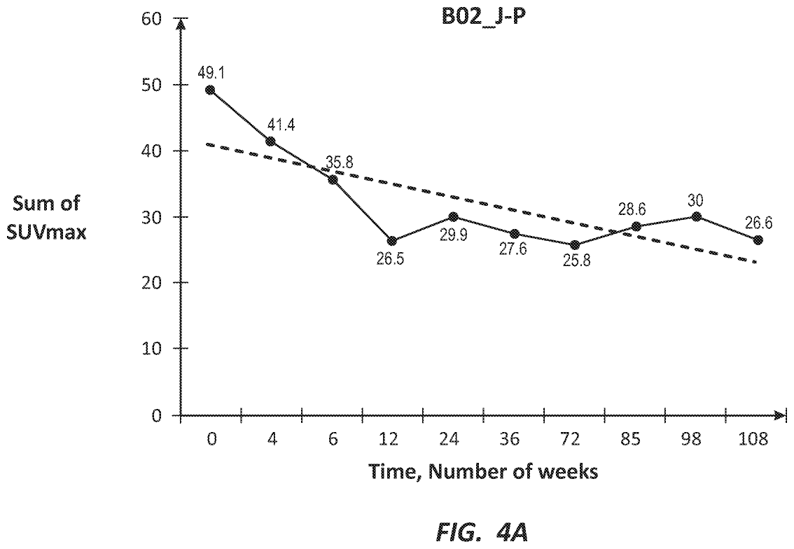

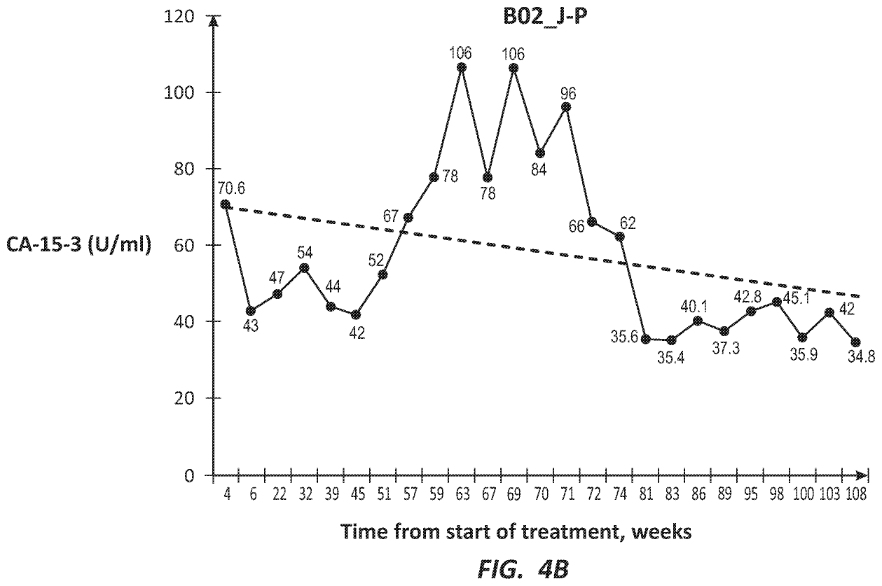

This individual is a 47 year old white female with moderately differentiated ductal carcinoma of breast and extensive bone metastases including 2nd and 5th lateral ribs, T3. T5, T6, T9, T10 thoracic, L1, L3, L4, L5 lumbar vertebrae, and right femoral head. The patient was treated for 72 weeks at Bruckner Oncology Clinic with DeltaRex-G 3×10¹¹ cfu i.v. three times a week for four weeks followed by a two-week rest period. Subsequently, she was treated in Manila with DeltaRex-G and DeltaVax, (The GeneVieve Protocol: Ignacio et al. (2018), Clinics in Oncology 3:1537). See FIGS. 4A and 4B.

TABLE 1

| Age (years) | |
|---|---|
| Median | 58.5 |
| Range | 33-77.0 |
| Gender | |
| Female | 20 (100%) |
| Race | |
| White | 18 (90%) |
| Asian | 2 (10%) |
| Receptor | |
| ER+ | 11 (55%) |
| HER2-Neu+ | 1 (5%) |
| Triple Neg. or NOS | 8 (40%) |
| Disease Stage | |
| Metastatic | 20 (100%) |
| Non-Metastatic | — |
| Performance Score | |
| 0 | — |
| 1 | 20 (100%) |
| # of Previous ChemoRx Regimens | |
| Median | 3 |
| Range | 1-12 |

TABLE 2
Listing of Patients with Drug-Related Adverse Events

| | | MedRA | | |
|---|---|---|---|---|
| Patient | MedRA | Preferred | Dose | Toxicity |
| ID | System Organ Class | Term | Level | Grade |
| 007DGA | General disorders and | Chills | II | 1 |
| | administration site | | | |
| | conditions | | | |
| | Skin and subcutaneous | Pruritus | II | 1 |
| | tissue disorders | | | |
| B01-M- | Skin and subcutaneous | Rash | III | 3 |
| M | tissue disorders | pruritic | | |
| B02-J-P | Nervous system disorders | Dysgeusia | III | 1 |
| B04-RJF | Nervous system disorders | Dysgeusia | IV | 1 |
| B01-M-M | Nervous system disorders | Dysgeusia | III | 2 |
| | Skin and subcutaneous | Dry skin | III | 2 |
| | tissue disorders | | | |
| | General disorders and | Hot flush | III | 2 |
| | administration site | | | |
| | conditions | | | |

Notes:
all drug-related AEs were nonserious; toxicity grade according to NCI-CTCAE.

TABLE 3
Summary of Responses

| | Dose Level^{a} | | | |
|---|---|---|---|---|
| Category | 0-II | III | IV | All |
| mITT Patients^{b} | N = 6 | N = 5 | N = 6 | N = 17 |
| Median tumor burden | 33.8 | 73.9 | 31.0 | N.D. |
| (# cells × 10e9) | | | | |
| Median Cum. Dose | 53 | 54 | 120 | N.D. |
| (×10e11 cfu) | | | | |
| Response by | 5SD; 1PD | 4SD; 1PD | 4SD; 2PD | 13SD; 4PD |
| RECIST | | | | |
| Response by PET | 2PR, 3SD, 1PD | 1PR, 4SD | 4SD, 2PD | 3PR, 11SD, 3PD |
| Response by CHOI | 3PR, 3SD | 4SD, 1PD | 4SD, 2PD | 3PR, 11SD, 3PD |
| Median PFS (mo) by | | | | |
| RECIST | 3.5 | 1.25 | 2 | 3 |
| Median OS (mo) | 25.6 | 6.75 | 21 | |
| ITT population^{c} | N = 7 | N = 7 | N = 6 | N = 20 |
| Median OS (mo)^{c} | 33.0 | 5.5 | 21.0 | 20.0 |
| % OS | | | | |
| 1 year | 71.4% | 28.6% | 83.0% | 60%^{d} |
| # Alive^{d} | 1/7 | 2/7 | 5/6 | 8/20 |

Abbreviations:
ITT, intent-to-treat;
mITT, modified ITT;
Cum, cumulative;
mo, month;
ND, not determined;
RECIST., Response Evaluation Criteria in Solid Tumors;
PET, positron emission tomography;
Choi, modified RECIST as described by Choi et al.
PFS, progression-free survival;
OS, overall survival
^{a}Dose Level 0 = 1 × 10e11 cfu twice per week (BIW); Dose Level I = 1 × 10e11 cfu three times per week (TIW); Dose Level II = 2 × 10e11 cfu TIW; Dose Level III = 3 × 10e11 cfu TIW; Dose Level IV = 4 × 10e11 cfu TIW.
^{b}mITT population was defined as all patients who received at least one cycle (4 weeks) of DeltaRex-G and had a follow-up PET CT scan
^{c}ITT population was defined as all patients who received at least one infusion of Delta Rex-G
^{d}Among patients with bone metastasis only, OS was 100% at 2 years

Case 2: Long Term (10-Year) Survivor with Pure Bone Metastases:

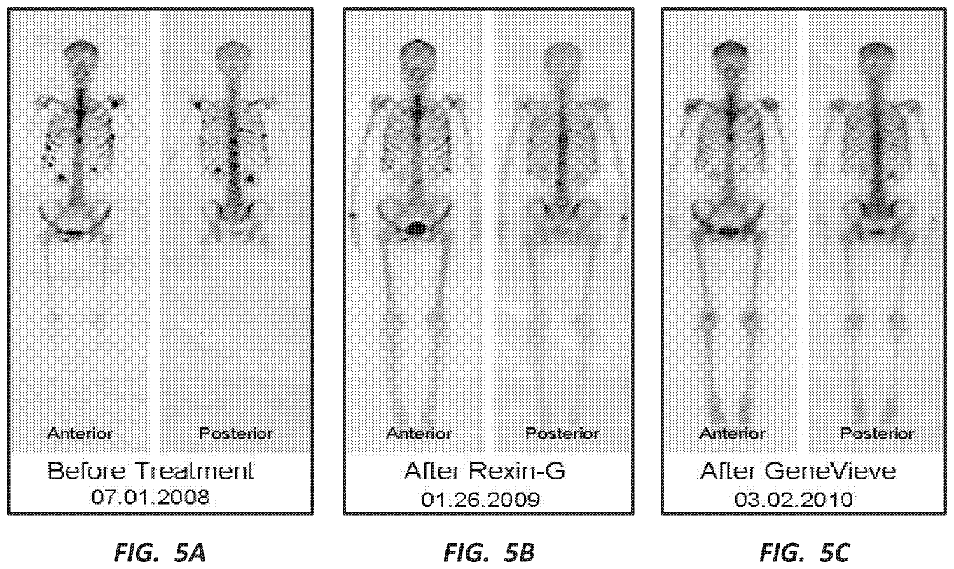

This individual is a 60 year old Filipino female with ductal carcinoma of breast and extensive bone metastases. The patient was treated in Manila with DeltaRex-G 3×10¹¹ cfu i.v. three times a week for four weeks followed by a two-week rest period, followed by DeltaVax under the GeneVieve Protocol. Bone scans show progressive regression of bone metastases during treatment with the GeneVieve Protocol. See FIG. 5.

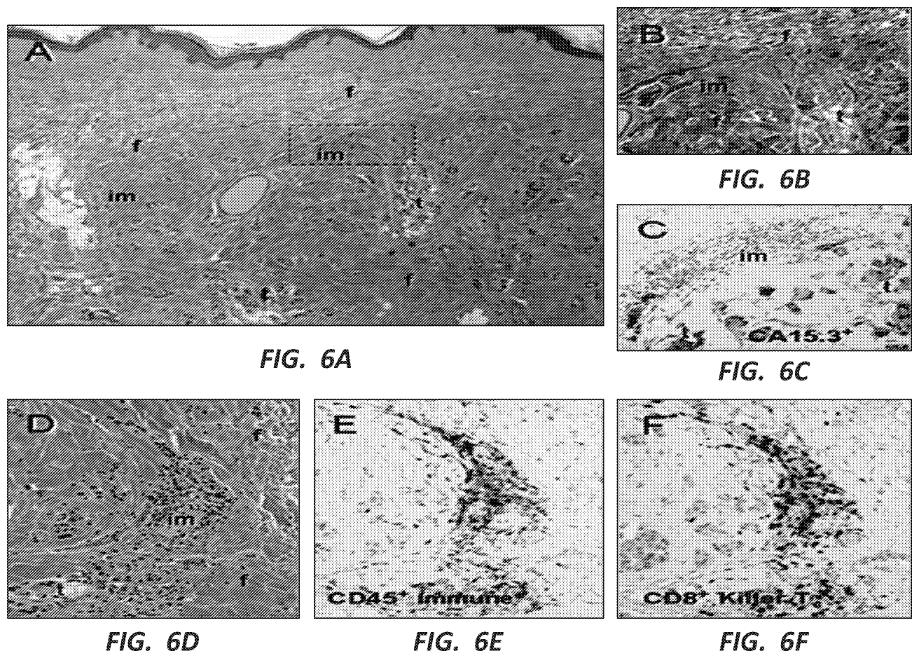

Conclusions: (1) DeltaRex-G therapy is uniquely safe, (2) exhibits antitumor activity particularly in patients with pure bone metastases, (3) PET-CT is a more sensitive indicator of early tumor responses to DeltaRex-G and will be used in planned Phase 2 studies, and (4) enhanced immune cell trafficking in the TME indicate that DeltaRex-G may be combined with other cancer therapy, including immunotherapy, and may prove to be a biochemical and/or antigen modulator. See FIGS. 4-6.

### Example 2

The inventors reviewed long-term data of patients with advanced chemotherapy-resistant malignancies, previously-treated patients with two tumor-targeted retrovectors: (1) encoding cytotoxic dominant negative cyclin G1, DeltaRex-G, and (2) encoding cytokine GM-CSF plus the suicide gene HSV-TK, DeltaVax (formerly Reximmune-C). Results: Ninety-nine patients received >5,000 intravenous infusions of DeltaRex-G; another 16 patients received 288 intravenous infusions of DeltaRex-G+96 infusions of DeltaVax followed by valacyclovir p.o. No therapy-related bone marrow suppression, organ dysfunction or delayed treatment related adverse events were observed. Survival analysis showed 5.0% 10-year overall survival rate for patients who received DeltaRex-G alone, and 18.8% for DeltaRex-G+DeltaVax. Table 4 lists the cancer type, name of targeted gene therapy/immunotherapy, treatment outcome, and reference source.

TABLE 4

| | Targeted Gene | | |
|---|---|---|---|
| | Therapy, Route of | | |
| Cancer Type | Delivery | Treatment Outcome | Reference |
| Pancreatic | DeltaRex-G, | Alive, >10 years, no | Chawla et al., |
| adenocarcinoma, | intravenous | evidence of disease, no | 2019; personal |
| metastatic to lymph | | further cancer therapy | communication |
| node, liver and | | | |
| peritoneum, resistant | | | |
| to gemcitabine and | | | |
| 5FU | | | |
| Osteosarcoma, | DeltaRex-G, | Alive, >10 years, no | AI-Shihabi et al., |
| metastatic to lung, | intravenous | evidence of disease, no | 2018; Chawla et |
| chemotherapy- | | further cancer therapy | al., 2016; |
| resistant | | | personal |
| | | | communication |
| Malignant peripheral | DeltaRex-G, | Alive, >10 years, no | AI-Shihabi et al., |
| nerve sheath tumor, | intravenous | evidence of active | 2018; Kim et al., |
| metastatic to lung, | | disease, no further | 2017; personal |
| chemotherapy- | | cancer therapy | communication |
| resistant | | | |
| Osteosarcoma, | DeltaRex-G, | Alive, >10 years, no | AI-Shihabi et al., |
| metastatic to lung, | intravenous | evidence of disease, no | 2018; personal |
| lymph nodes, pelvic | | further cancer therapy | communication |
| soft tissue, | | | |
| chemotherapy- | | | |
| resistant | | | |
| Intraductal carcinoma | DeltaRex-G, | Alive, >10 years, no | Bruckner et al., |
| of breast, recurrent, | intravenous | evidence of active | 2019; personal |
| chemotherapy- | | disease, no further | communication |
| resistant | | cancer therapy | |
| B-cell lymphoma, | DeltaRex-G + | Alive, 10 years, no | Ignacio et al., |
| cervical lymph node, | DeltaVax, | evidence of disease, no | 2018; personal |
| metastatic to liver and | intravenous | further cancer therapy | communication |
| pancreas, | | | |
| chemotherapy- | | | |
| resistant | | | |
| Chondroblastic | DeltaRex-G + | Alive, 10 years, no | Ignacio et al., |
| osteosarcoma of | DeltaVax, | evidence of active | 2018; personal |
| maxilla, locally | intravenous | disease, no further | communication |
| advanced, | | cancer therapy | |
| unresectable | | | |
| Intraductal carcinoma | DeltaRex-G + | Alive, >10 years, with | Ignacio et al., |
| of breast metastatic to | DeltaVax, | disease progression, on | 2018; personal |
| bone, chemotherapy | intravenous | capecitabine for liver | communication |
| resistant | | metastases/no late | |
| | | adverse event | |

Case 1: Pancreatic adenocarcinoma metastatic to lymph node, liver and peritoneum. Patient 018 is a 72 year old white female who had been initially diagnosed with non-metastatic, poorly differentiated adenocarcinoma of the pancreas, underwent a Whipple's resection with postoperative radiation therapy, and received chemotherapy with 5-FU and gemcitabine for one year. A year later, she presented with hepatic and lymph node metastases (target lesions), and mesenteric stranding indicative of peritoneal carcinomatosis (non-target lesions) with a rising serum CA19.9. She was advised to receive chemotherapy, but decided to participate in the Phase I/II study using DeltaRex-G.

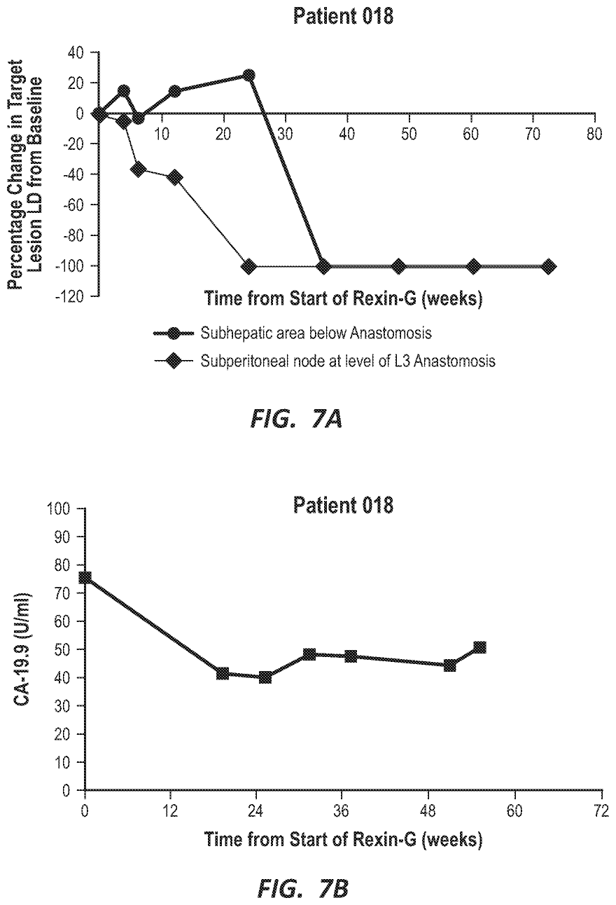

The patient completed a total of 17.9 months of therapy with DeltaRex-G and did not achieve CR until Week 36 of treatment; this patient has remained in CR at the time of study completion. Notably, when examined separately, the two target lesions were found to have different disappearance profiles. As shown in FIG. 7A, the lymph node metastasis (black line) decreased more rapidly, starting at Week 6, than the hepatic metastasis (gray line), which increased in size before completely resolving by Week 36. Serum levels of the tumor marker CA-19.9 (FIG. 7B) decreased by 45%, from 76 to 42 U/mL (normal level is <37 U/mL) by Week 19, and then remained relatively constant thereafter.

The patient, now 82 years old, received no additional chemotherapy or alternative treatment after discontinuation of DeltaRex-G therapy and remains in sustained remission with no evidence of disease or late onset adverse events as of April 2019.

Case 2: This 39 year-old female with osteosarcoma metastatic to lung was treated with DeltaRex-G, 3×10¹¹ cfu three times a week for six months, followed by resection of residual tumors, and then given another six months of DeltaRex-G therapy (Chawla et al. (2009), Molecular Therapy 17(9):1651-7).

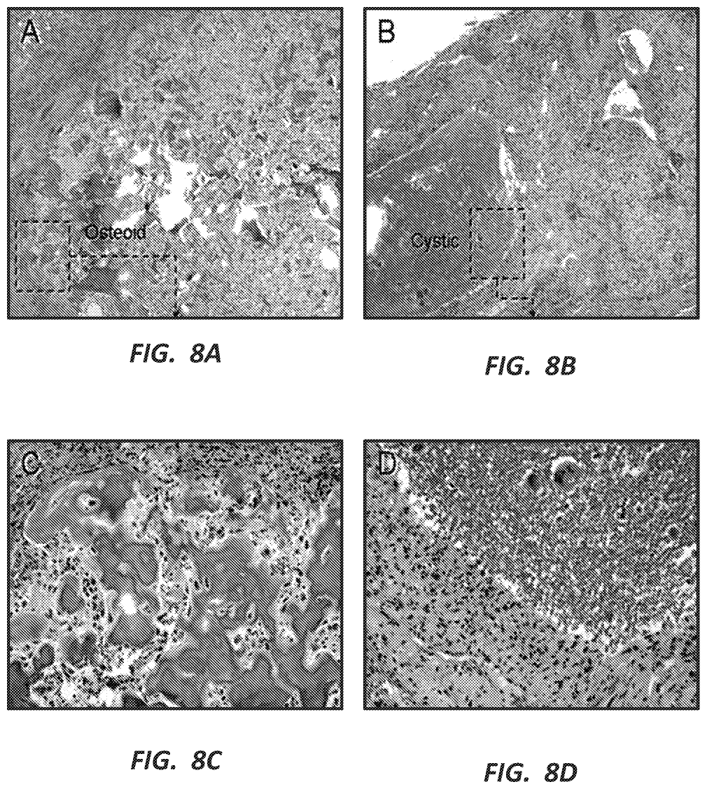

Ten years later, the most recent CT scan showed no evidence of osteosarcoma and the patient, now 49 years old, enjoys sustained remission (Al-Shihabi et al., 2018). See FIG. 8, which illustrates representative H&E stained tissue sections from resected residual lung nodules in this DeltaRex-G treated patient. Left side (A and C): 2.5 cm cystic mass with organized clot and rare osteosarcoma cells. Right side (B and D): 0.9 cm calcified tumor mass with reparative fibrosis, immune infiltrate and osteosarcoma cells with rare mitosis.

Of note, the 2.5 cm tumor had increased in size and was diagnosed as progressive disease (PD) by RECIST but a PR by PET criteria. This finding suggests that RECIST criteria may not be a reliable indicator of tumor response to DeltaRex-G treatment in osteosarcoma.

Case 3: Malignant Peripheral Nerve Sheath Tumor (MPNST), parotid gland, metastatic to lung, chemotherapy resistant.

This 15 year old white female with advanced chemotherapy-resistant MPNST participated in a Phase 1/2 clinical trial using i.v. DeltaRex-G for advanced bone and soft tissue sarcoma in March 2008.

She received Dose Level 3 of DeltaRex-G, 3×10¹¹ cfu, three times per week as an outpatient.

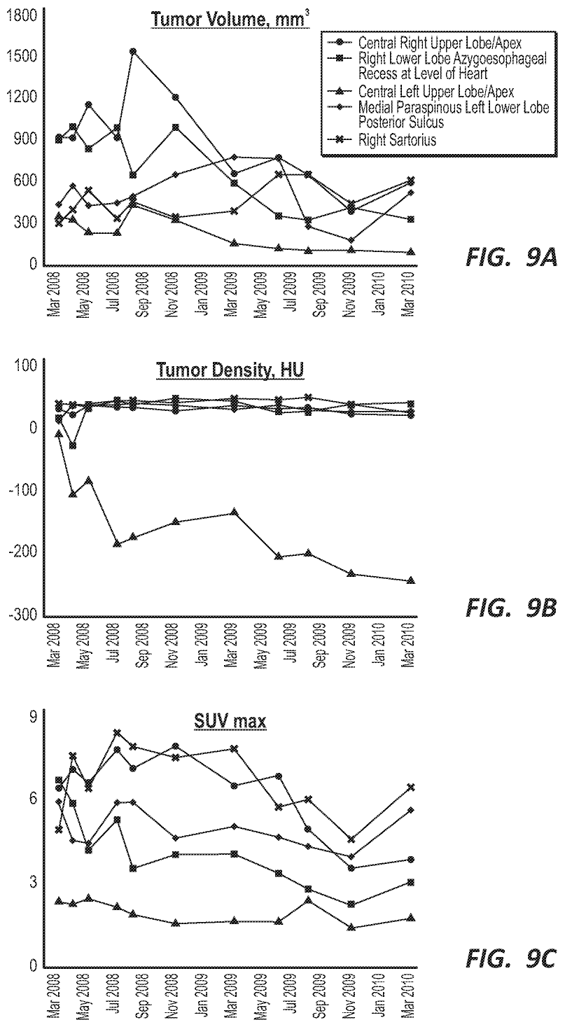

Objective tumor responses were evaluated by a number of parameters including RECIST v1, tumor volume, mm³ (length×width²×0.52), tumor density in Hounsfeld Units (HU) and SUVmax by PET. By RECIST v1 and other radiologic parameters, the patient experienced sustained disease control over 28 months (FIG. 9A-9C).

The patient received a total of 205 DeltaRex-G vector infusions with minimal toxicity and no serious adverse events.

In June 2010, the patient had a follow-up PET/CT scan which was compared to the images taken before treatment. The radiology report stated that there was overall significant improvement of the patient's pulmonary metastases, with all but one nodule being either markedly improved in size or resolved. The right pleural effusion and previous significant ascites had both resolved.

The patient, now 25 years old, has no evidence of active disease on no further therapy.

These data indicate that tumor-targeted gene delivery in vivo, represented by cytocidal DeltaRex-G, with or without immuno-stimulatory DeltaVax, has induced prolonged (>10 years) sustained remissions in cancer patients presenting with advanced chemotherapy-resistant solid and hematologic malignancies—plausibly due to safety, selectivity, and immune modulation. While the curative potential of precision targeted genetic medicine necessarily remains an academic question; it is clear that these initial long-term, cancer-free (>10 year) survivors represent a major milestone in both cancer therapy and immunotherapy. Phase 2-3 clinical trials are planned for these hard-to treat malignancies.

### Example 3

Metastatic cancer is associated with, hitherto, an invariably fatal outcome. DeltaRex-G, a CCNG1 inhibitor, has induced long-term (>12 years) survival in patients with metastatic sarcoma, lymphoma, and cancer of the pancreas and breast, with no dose-limiting toxicity or long-term adverse events. Purpose: To evaluate the incidence of genetic mutations along the CCNG1 pathway for identification of patients who are likely to benefit as long term survivors (>10 years) from DeltaRex-G tumor-targeted gene therapy. Methods: Four hundred fifty-one (451) patients were treated at the Cancer Center of Southern California/Sarcoma Oncology Center from October 2019 to April 2020. The archived tumors of one hundred sixty-three (163) of these patients underwent molecular profiling. The data were reviewed for genetic mutations relevant to the CCNG1 pathway. Results: Thirty-three of 163 (20.2%) of patients had genetic mutations along the CCNG1 pathway. Ten (6.1%) patients had MDM2 gene amplification; 2 (1.2%) patients had MYC mutation with 1 patient having both MYC and MDM2 gene amplification; 21 (12.9%) patients had TP53 mutation/loss; 1 patient had CCNG1 overexpression. Only one patient was tested for CCNG1 expression. The table below summarizes the collected molecular profiles with the aforesaid mutations. Importantly, of those with genetic mutations, 6% had a MYC mutation.

TABLE 5

| Sarcoma Subtype | Meta- | | | | | | |
|---|---|---|---|---|---|---|---|
| (n = 33) | static | MYC | MDM2 | CDK2 | CDK5 | TP53 | CCNG1 |
| Dedifferentiated | No | — | 5 | — | — | — | — |
| liposarcoma (n = 5) | | | | | | | |
| Dedifferentiated | Yes | — | 1 | — | — | — | — |
| liposarcoma (n = 1) | | | | | | | |
| Well-differentiated | No | — | 1 | — | — | — | — |
| liposarcoma (n = 1) | | | | | | | |
| Undifferentiated | No | — | — | — | — | 2 | — |
| pleomorphic sarcoma | | | | | | | |
| (n = 2) | | | | | | | |
| Undifferentiated | Yes | — | 1 | — | — | 3 | — |
| pleomorphic sarcoma | | | | | | | |
| (n = 4) | | | | | | | |
| Leiomyosarcoma (n = 2) | No | — | — | — | — | 2 | — |
| Leiomyosarcoma (n = 9) | Yes | — | — | — | — | 8 | 1 |
| Myxofibrosarcoma (n = 1) | No | 1 | 1 | — | — | — | — |
| Myxofibrosarcoma (n = 1) | Yes | — | — | — | — | 1 | — |
| Desmoplastic small round | No | — | 1 | — | — | — | — |
| cell tumor (n = 1) | | | | | | | |
| Adenosarcoma (n = 1) | Yes | 1 | — | — | — | — | — |
| Carcinosarcoma (n = 1) | No | — | — | — | — | 1 | — |
| Carcinosarcoma (n = 1) | Yes | — | — | — | — | 1 | — |
| Pleomorphic | Yes | — | — | — | — | 1 | — |
| rhabdomyosarcoma (n = 1) | | | | | | | |
| Sarcoma, not otherwise | Yes | — | — | — | — | 2 | — |
| specified (NOS) (n = 2) | | | | | | | |

Conclusion: Taken together, these data indicate that the frequency of genetic mutations along the CCNG1 pathway in sarcoma is not uncommon. Further, these mutations could identify patients who are likely to benefit from DeltaRex-G therapy, and may serve as biomarkers for long-term survival/cure, plausibly via eradication of the cancer stem cell as in patients with MYC mutation/amplification. Consistent with these findings, 3 of 54 (6%) of patients treated with DeltaRex-G in a US-based Phase 1 and Phase 2 study for metastatic chemotherapy-resistant sarcoma are long-term survivors (>12 years) with no further cancer therapy. This corresponds to the frequency of MYC mutations in sarcoma.

All references cited in this disclosure are incorporated by reference in their entirety.

1. A method of treating an individual having a chemotherapy-resistant malignancy comprising,
a. administering a therapeutically effective amount of a tumor-targeted retrovector to the individual, wherein the retrovector encodes a cytocidal dominant-negative cyclin G1 construct.

2. The method of claim 1, wherein the retrovector is DeltaRex-G.

3. The method of claim 1, wherein the chemotherapy-resistant malignancy is a soft tissue sarcoma, osteosarcoma, or pancreatic adenocarcinoma.

4. The method of claim 1, further comprising the step of administering one or more additional cancer therapies.

5. The method of claim 4, wherein the one or more additional cancer therapies comprise an immunotherapy.

6. The method of claim 4, wherein the one or more additional cancer therapies comprise a tumor-targeted retrovector encoding a GM-CSF protein.

7. The method of claim 6, wherein the tumor-targeted retrovector encoding a GM-CSF protein is Reximmune-C.

8. The method of claim 1, wherein the retrovector is administered intravenously.

9. The method of claim 8, wherein the retrovector is administered at a dose of greater than about 10¹⁰ cfu.

10-21. (canceled)

22. A method for improving the long-term survival of an individual with a metastatic tumor, the method comprising:
a. identifying whether the metastatic tumor exhibits at least one of CCNG1 overexpression, Myc amplification, or genetic mutations along the CCNG1 axis including IGH-CCNG1 gene rearrangement; and,
b. administering a high-dose, therapeutically effective amount of a CCNG1 inhibitor to the individual if the metastatic tumor exhibits at least one of CCNG1 overexpression and Myc amplification, or adding an alternative therapy if the tumor does not exhibit at least one of CCNG1 overexpression and Myc amplification or genetic mutations along the CCNG1 axis.

23. The method of claim 22, comprising administering the high-dose, therapeutically effective amount of the CCNG1 inhibitor to the individual for at least three weeks.

24. The method of claim 23, comprising administering the high-dose, therapeutically effective amount of the CCNG1 inhibitor to the individual for up to 24 months.

25. The method of claim 22, comprising administering the CCNG1 inhibitor once weekly, twice weekly, or three times weekly, daily or as continuous intravenous infusion (CIV).

26. The method of any of claims 22, wherein the CCNG1 inhibitor is a tumor-targeted retrovector encoding a cytocidal dominant-negative cyclin G1 construct.

27. The method of claim 26, wherein the CCNG1 inhibitor is DeltaRex-G.

28. The method of claim 26, comprising administering the CCNG1 inhibitor three times per week in an amount from about 1×10¹¹ cfu to about 5×10¹¹ cfu per administration.

29-32. (canceled)

33. A method for preventing occurrence of a secondary malignancy in an individual having a tumor load, the method comprising:
a. administering a CCNG1 inhibitor to the individual in a therapeutically effective total dose over a treatment period of up to 15 weeks; and
b. administering no further anti-cancer therapy to the individual after the treatment period.

34. The method of claim 33, wherein the CCNG1 inhibitor is a tumor-targeted retrovector encoding a cytocidal dominant-negative cyclin G1 construct.

35. The method of claim 34, wherein the CCNG1 inhibitor is DeltaRex-G.

36. The method of claim 34, wherein the therapeutically effective total dose comprises about 1000 cfu multiplied by the tumor load.

37-66. (canceled)
